(19) United States
(12) Patent Application Publication
(10) Pub. No.: US 2004/0252846 A1
(43) Pub. Date: Dec. 16, 2004

(54) Noise reduction apparatus

(75) Inventors: Yoshiya Nonaka, Kawagoe-shi (JP); Nobuo Tarui, Kawagoe-shi (JP); Takamasa Yamaguchi, Kawagoe-shi (JP); Akihiro Denda, Kawagoe-shi (JP); Ryoji Sugino, Tokyo (JP)

Correspondence Address: MCGINN & GIBB, PLLC, 8321 OLD COURTHOUSE ROAD, SUITE 200, VIENNA, VA, 22182-3817, US

(73) Assignees: Pioneer Corporation, Tokyo (JP); TechExperts Incorporation, Tokyo (JP)

(21) Appl. No.: 10/862,310

(22) Filed: Jun. 8, 2004

(30) Foreign Application Priority Data
Jun. 12, 2003 (JP) 2003-168404

(51) Int. Cl. A61F 11/06; G10K 11/16; H03B 29/00

(52) U.S. Cl. 381/71.4; 381/71.8

(57) ABSTRACT

A noise reduction apparatus is capable of suppressing deterioration of an aural sensing level due to noise existing in a sound field. The apparatus synthesizes a signal obtained by adding a predetermined delay to a sound field noise and reversing and amplifying the signal with an electric signal from a sound reproduction system to obtain a sound pressure output by using a first signal processing part. On the other hand, the apparatus synthesizes a signal obtained by delaying an electric signal from the sound reproduction system by a predetermined period and reversing and amplifying the signal with an electric signal obtained from a sound pressure in the proximity of a concha of a listener to extract a residual noise component by using a second signal processing part. The apparatus controls the delay amounts and the gains set by the first and second signal processing parts so as to minimize an extraction signal.

### BACKGROUND OF THE INVENTION

1. Field of the Invention

The present invention relates to a noise reduction apparatus in a sound reproduction system mounted in a moving vehicle, for example.

2. Description of the Related Art

When a passenger listens to musical pieces and news broadcasting by using a sound reproduction system such as a compact disk player, an FM radio, or the like, mounted in a moving vehicle such as a wheeled vehicle, a ship, an aircraft, etc, cruising noise of the moving vehicle may become annoying. In particular, when the moving vehicle is cruising at a high speed, such a noise becomes extremely large and the musical piece and the speech reproduced and outputted from the sound reproduction system are masked in some case by the noise.

To reduce the influences of the noise, Japanese Patent Kokai No. H5-46182 (Patent Reference 1) discloses a technology for reducing noise.

One of such prior art technologies includes an apparatus that collects noise through a microphone, for example, applies a predetermined delay, reverses and amplifies the noise so delayed, electrically adds it to a musical piece signal from a sound reproduction system and acquires an acoustic output. In this case, when such an acoustic output is outputted from a loud speaker, the acoustic output from the loud speaker is spatially synthesized with a noise occurring actually in a sound field space and the noise is thus cancelled.

Another prior art technology discloses an apparatus that collects noise through a microphone, measures its volume level and adjusts a reproduction output level from a sound reproduction system in accordance with a measurement level. In other words, the reproduction output level of a musical piece, etc is increased with the increase of the noise level to relatively reduce a mask effect by the noise.

In these prior art technologies, however, a noise cancellation operation can be insufficient and fail to provide satisfaction to a listener listening to the musical piece from the sound reproduction system inside a cabin of a moving vehicle. In other words, because these prior art technologies have a main target to cancel the cruising noise collected through the microphone, they cannot effectively cancel noise signal components such as musical pieces other than the musical piece that is reproduced inside the cabin and conversation inside the cabin. As a result, the acoustic output of the musical piece played back contains these noise signal components and fails to fully satisfy the listener.

It is an object of the invention to provide a noise reduction apparatus capable of suppressing deterioration of a sound hearing level due to noise existing in a sound field.

### SUMMARY OF THE INVENTION

According to an aspect of the invention, there is provided a noise reduction apparatus in a sound reproduction system for reproducing and outputting sound to a sound field based on electric audio signals, comprising first and second microphones arranged in the sound field; a first signal processing part for executing a delay processing and an inversion amplification processing for a first sound field electric signal acquired through the first microphone on the basis of a first delay instruction value and a first gain instruction value; a reproduction output part for reproducing and outputting a superimposed signal acquired by superimposing the electric audio signal to an output signal of the first signal processing part; a second signal processing part for executing a delay processing and an inversion amplification processing for the electric audio signal on the basis of a second delay instruction value and a second gain instruction value; a signal superimposing part for superimposing a second sound field electric signal acquired through the second microphone with an output signal from the second signal processing part; and a feedback control part for controlling the first and second delay instruction values and the first and second gain instruction values in accordance with the magnitude of the output signal from the signal superimposing part.

### BRIEF DESCRIPTION OF THE DRAWINGS

FIG. 1 is a block diagram showing a cruising noise reduction apparatus inside a moving vehicle according to an embodiment of the invention;

FIG. 2 is a flowchart showing an operation of an initial value setting processing in the cruising noise reduction apparatus shown in FIG. 1; and

FIG. 3 is a flowchart showing an operation of a noise cancellation processing in the cruising noise reduction apparatus shown in FIG. 1.

### DETAILED DESCRIPTION OF THE INVENTION

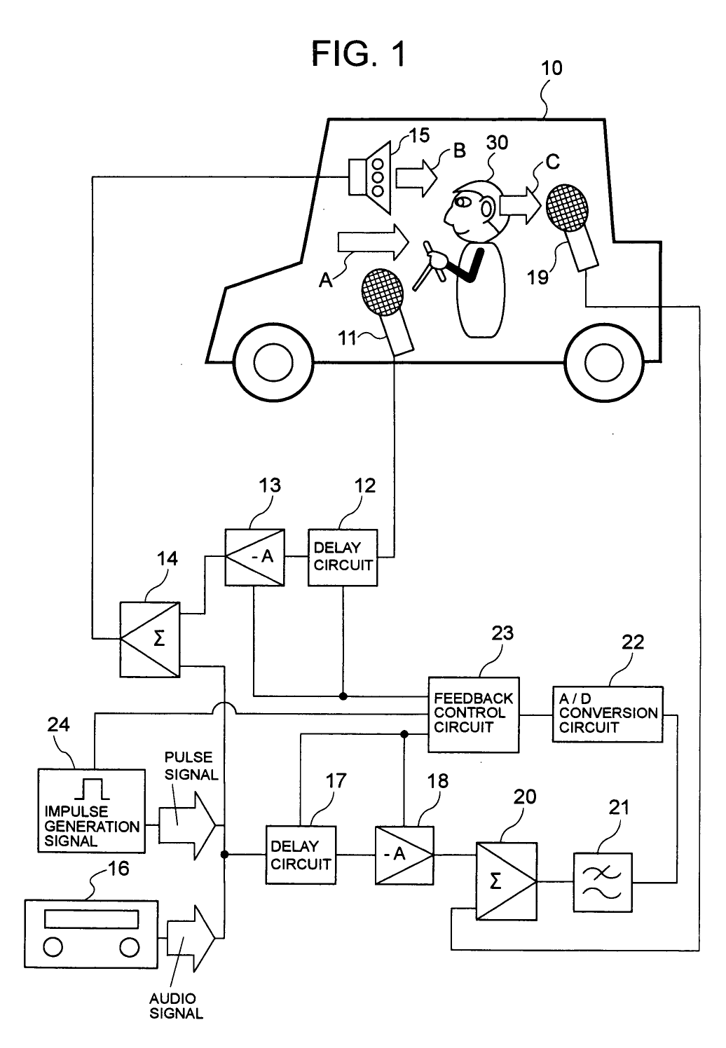

A noise reduction apparatus in a car-mounted sound reproduction system according to an embodiment of the invention is shown in a block diagram of FIG. 1.

Referring to FIG. 1, a microphone 11 as a first microphone (hereinafter called “noise mic 11”) is installed inside a cabin of a car 10, for example, and picks up various cruising noises such as engine noise generated from the car 10 and cruising noise of tires.

A delay circuit 12 is a circuit that delays the cruising noise signal by a predetermined time as a first sound field electric signal obtained through the noise mic 11. An inversion amplification circuit 13 reverses a phase of an output signal from the delay circuit 12 and amplifies the output signal at a predetermined degree of amplification, that is, a gain. Incidentally, the delay amount in the delay circuit 12 and the gain in the inversion amplification circuit 13 can be freely adjusted in accordance with a first delay instruction value and a first gain instruction value supplied from a later-appearing feedback control circuit 23, respectively. These delay circuit 12 and inversion amplification circuit 13 constitute a first signal processing part.

A signal addition circuit 14 is an addition circuit that electrically adds and synthesizes the output signal from the inversion amplification circuit 13 to and with a musical piece signal from a later-appearing sound reproduction system 16.

A loud speaker 15 operates as a reproduction output part that converts the electric audio signal outputted from the signal addition circuit 14 to an acoustic output and outputs it inside the cabin of the car 10. Incidentally, the number of loud speakers is not particularly limited to one and a construction including a plurality of loud speakers such as a surround system stereo system may be used, too.

The sound generation apparatus 16 is a car-mounted type sound generation apparatus such as a compact disk player, an FM radio, etc, and outputs electric audio signals such as the musical piece signals to the signal addition circuit 14 and to the delay circuit 17.

The delay circuit 17 imparts a predetermined delay to the electric audio signals such as the musical piece signal supplied from the sound reproduction system 16. An inversion amplification circuit 18 reverses a phase of an output signal from the delay circuit 17 and amplifies the output signal at a predetermined degree of amplification, that is, a gain. Incidentally, the delay amount in the delay circuit 17 and the gain in the inversion amplification circuit 18 can be freely adjusted in accordance with a second delay instruction value and a second gain instruction value supplied from a later-appearing feedback control circuit 23. These delay circuit 17 and inversion amplification circuit 18 constitute a second signal processing part.

A microphone 19 as a second microphone (hereinafter called “ear mic 19”) is a high directional microphone fitted preferably to a part in the proximity of a concha of a listener 30 listening to a musical piece inside the cabin. In other words, the ear mic 19 is preferably disposed at a position closer to a driver's seat inside the cabin than the noise mic 11, moreover closer to the concha of the driver. Consequently, the ear mic 19 can collect the sound signals existing in the sound field in the proximity of the concha of the user, that is, the sound signals that may be perceived by the hearing sense of the user.

Incidentally, the number of each of the noise mic 11 and the ear mic 19 is not limited to the example shown in FIG. 1. For example, it is possible to use a plurality of mics for each of the mics and to use a mean value of the electric signals acquired through each mic as the input signal from each mic.

A signal addition circuit 20 is an addition circuit that electrically adds and synthesizes the output signal from the inversion amplification circuit 18 and the input signal as the second sound field signal acquired through the ear mic 19 and operates as signal superimposing means. A low-pass filter 21 (hereinafter merely called “LPF 21”) is a low-pass filter that extracts noise signal components contained in the output signal from the signal addition circuit 20.

Incidentally, the embodiment shown in FIG. 1 uses the low-pass filter (LPF 21) for passing only frequency components below hundreds of Hertz as a constituent element for extracting the noise components contained in the output signal from the signal addition circuit 20. This is directed to remove ordinarily the cruising noise of the car containing noises of a low frequency band. Therefore, the embodiment of the invention is not limited thereto and a constituent element suitable for each embodiment may of course be used for extracting the noise components corresponding to the LPF 21.

An analog/digital conversion circuit 22 (hereinafter merely called “ADC 22”) converts the output signal from the LPF 21 at a predetermined sample frequency to a digital data having a predetermined bit length. The digital data output from the ADC 22 is supplied to the feedback control circuit 23.

The feedback control circuit 23 as a feedback control part mainly includes a microcomputer, a memory circuit such as ROM and RAM and their peripheral circuits (none of them are shown in the drawing) and controls the apparatus shown in FIG. 1 as a whole. Incidentally the memory circuit stores various programs stipulating the operations of the present apparatus. Each of these programs is executed step-wise at a predetermined timing in synchronism with a clock signal provided to the feedback control circuit 23 and various kinds of operation processing of the present apparatus are executed. The feedback control circuit 23 outputs a predetermined delay instruction value, a gain instruction value and various control signals to each of the delay circuits 12 and 17, the inversion amplification circuits 13 and 18 and a later-appearing impulse generation circuit 24.

The impulse generation circuit 24 generates a pulse for measuring an impulse response inside the cabin of the car 10 in order to determine initial values of the delay amounts to be set to the delay circuits 12 and 17 and the gains to be set to the inversion amplification circuits 13 and 18. In other words, the impulse generation circuit 24 outputs a pulse signal at a predetermined timing on the basis of the instruction from the feedback control circuit 23. Incidentally, the impulse generation circuit 24 and the feedback control circuit 23 constitute an initial value setting part.

In the description given above, the explanation of a pre-amplification circuit for amplifying the input signal from each mic, a power amplification circuit for driving the loud speaker 15 and a power circuit for supplying a power source to each constituent element shown in FIG. 1 is omitted because they are not directly relevant to the embodiment of the invention.

In the description given above, each constituent element of the embodiment shown in FIG. 1 has been explained as hardware that exists discretely but the embodiment of the invention is not limited to such a construction. For example, the function of each of these constituent elements may be accomplished by software processing using operational devices such as a DSP.

Next, the principle of the operation of the noise reduction apparatus according to the embodiment will be explained.

Referring to FIG. 1, the sound pressure output B outputted from the loud speaker 15 contains a musical piece signal s from the sound reproduction system 16 and a noise cancel signal a′ which has an opposite phase to that of the noise A and to which a predetermined delay is added as expressed by the formula given below:

B=(s+a′)a cos

Incidentally, the term (s+a′)a cos is defined as expressing a conversion value of s+a′ as an electric signal to a sound pressure output.

Symbols B and A are spatially synthesized to a sound pressure C and reach the concha of the listener 30. In this case, the synthesis in the sound field space offsets the noise A and the sound pressure output A′ of the noise cancel signal a′ and only S as the sound pressure output of the musical piece signal s remains in the sound pressure C.

C=B+A=(s+a′)a cos+A=S+A′+A≈S

However, it is generally difficult to completely cancel the noise in such a sound field space and a residual noise E that cannot be cancelled fully remains in C. In other word, the musical piece signal component that should be handled as the noise in the sound pressure output B cannot be cancelled and the residual noise E remains.

Therefore, the sound pressure C0 expressed by the following equation reaches in practice the concha of the listener 30:

C0=E+S

This embodiment generates the musical piece cancel signal s′ which has the opposite phase to that of the musical piece signal s and to which a predetermined delay is added, by using the delay circuit 17 and the inversion amplification circuit 18. Next, the embodiment electrically synthesizes the electric signal [C0]elec acquired through the ear mic 19 and the musical piece cancel signal s′ by using the signal addition circuit 20. Incidentally, [C0]elec is defined as a conversion value of the sound pressure C0 to the electric signal.

Consequently, the musical piece signal s and the musical piece cancel signal s′ are offset with each other as represented by the following equation and only e that is the conversion result of the residual noise E to the electric signal appears in the output of the LPF 21 connected to the signal addition circuit 20:

[C0]elec+s′=[E+S]elec+s′=e+s+s′=e

To minimize the value of such a residual noise component e, the feedback control circuit 23 controls the values of the delay amounts and the gains to be supplied to the delay circuits 12 and 17 and to the inversion amplification circuits 13 and 18.

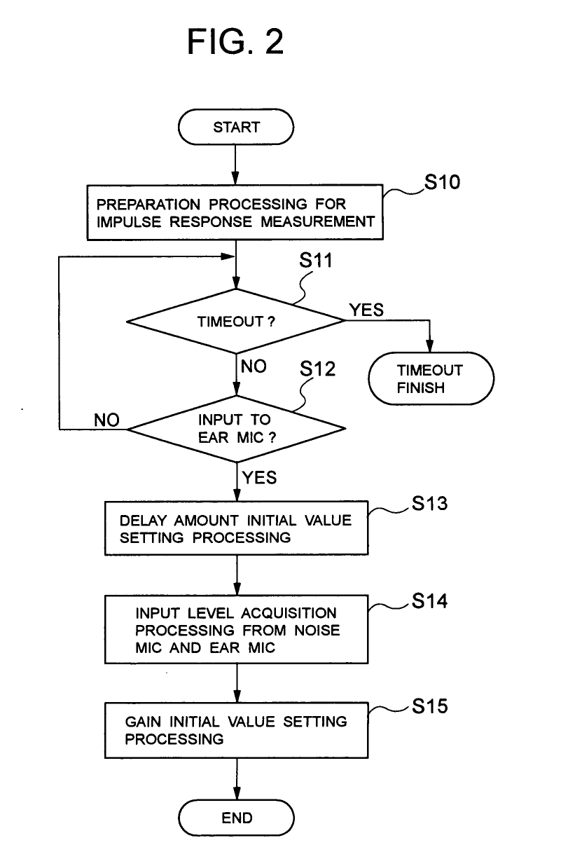

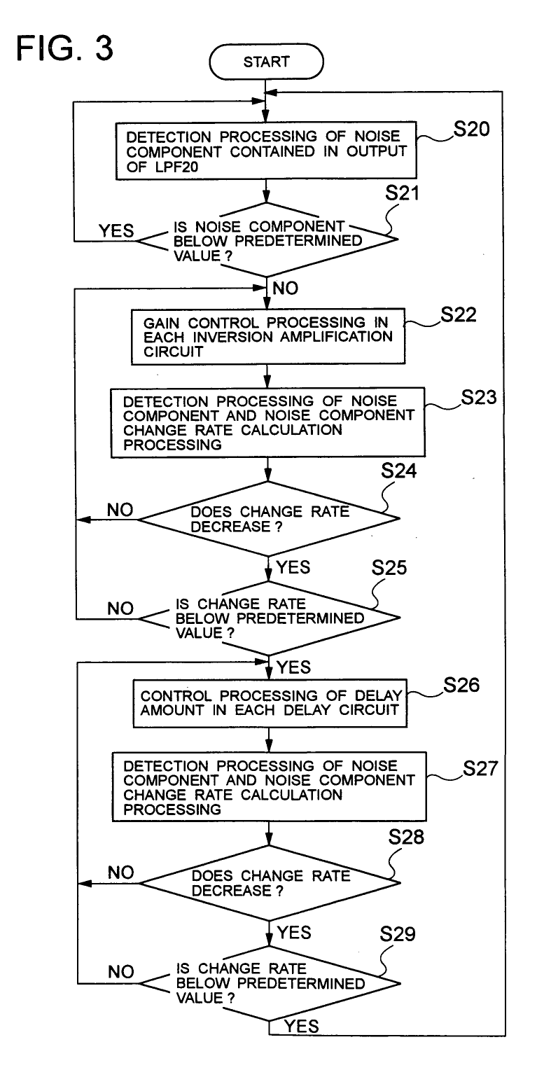

Next, a concrete operation of the noise reduction processing in this embodiment will be explained. Because this processing is mainly divided into the initial value setting processing and the noise cancellation processing, each of these processing will be explained with reference to the flowcharts shown in FIGS. 2 and 3, respectively.

First, the initial value setting processing will be explained with reference to the flowchart of FIG. 2. Incidentally, the initial value setting processing is the processing that sets the initial values of the delay amounts and the gains to be supplied to the delay circuits 12 and 17 and to the inversion amplification circuits 13 and 18 before the execution of the noise cancellation processing. Therefore, the initial value setting processing may well be actuated in synchronism with making of the power source of the sound reproduction system 16 or may well be actuated when the user of the apparatus pushes down a predetermined reset switch.

When the initial value setting processing shown in FIG. 2 is started, the feedback control circuit 23 first executes an impulse response measurement preparation processing in Step S10. The impulse response measurement preparation processing is a preparation processing that measures in advance an impulse response in the sound field space inside the car 10 and determines the initial values of the delay amounts to be set to the delay circuits 12 and 17.

In other words, the feedback control circuit 23 drives the impulse generation circuit 24 in Step S10, lets it generate the pulse for measuring the impulse response, operates a predetermined timer after storing the output timing of such a pulse and proceeds to the next Step S11.

The feedback control circuit 23 monitors the count value of the timer in Step S11, recognizes that any trouble occurs in the microphones and other constituent elements when time-out occurs after the passage of a predetermined set time and finishes the initial value setting processing shown in FIG. 2.

When time-out is not detected in Step S11, the feedback control circuit 23 proceeds to Step S12 and judges whether or not the signal by the measurement pulse reaches the ear mic 19. When the signal does not reach the ear mic 19, the flow returns to Step S11 and when it does, the flow proceeds to Step S13.

In Step S13, the feedback control circuit 23 first stores the input timing at which the signal reaches the ear mic 19. Next, the impulse response inside the car 10 is calculated from the time difference between the input timing and the output timing of the pulse stored in Step S10 and the delay amount of the audio signal during propagation is calculated.

To improve the accuracy of the impulse response measurement, it is also possible to measure in advance the noise in the proximity of the mic in Step S10, then to subtract such a measurement value from the input of the ear mic 19 and to detect the input timing of the pulse signal at the ear mic 19.

The feedback control circuit 23 makes a predetermined correction for the delay amount so calculated, determines the delay amounts required for the delay circuits 12 and 17 and sets these values as the initial values of the delay amounts of the respective circuits.

In the processing described above, it is possible not only to determine the initial values of the delay amounts but also to reproduce the waveform of the pulse from the component after the removal of the noise of the signal inputted to the ear mic and to determine the frequency transmission characteristics in the sound field space. It is further possible to determine the frequency transmission characteristics in the sound field space by using a white noise as a sample signal instead of using the pulse signal.

After finishing the setting processing of the initial values of the delay amounts in Step S13, the feedback control circuit 23 proceeds to the next Step S14 and measures the input level of each of the noise mic 11 and the ear mic 19. In the next Step S15, the feedback control circuit 23 calculates the gains necessary for the inversion amplification circuits 13 and 18 on the basis of the input levels measured in Step S14 and sets these values as the initial values of the gains of the respective circuits. After finishing the processing described above, the feedback control circuit 23 finishes the initial value setting processing shown in FIG. 2.

In the explanation given above, the initial values of the delay amount and the gain of the delay circuits and the inversion amplification circuits are determined by the measurement in the initial value setting processing but the embodiment of the invention is not particularly limited thereto. For example, correct measurement may be conducted in advance for typical car models to determine each initial value and the initial value so obtained is stored into the memory of the feedback control circuit 23 so that the user selects the car model in the initial value setting processing and the initial value can be set as the initial value of each of the delay amount and the gain.

Next, the operation of the noise cancellation processing will be explained with reference to the flowchart of FIG. 3. Incidentally, the noise cancellation processing may be executed in a predetermined time interval during the operation of the sound reproduction system 16 after the initial value setting processing is completed or may be executed whenever the change of the mode such as the change of a reproduction source or a volume operation occurs in the sound reproduction system 16.

First, in Step S20 in FIG. 3, the feedback control circuit 23 detects the residual noise component e contained in the output of the LPF 21 and judges whether or not the detection value is below a predetermined threshold value stored in the memory of the feedback control circuit 23 (Step S21).

When the detection value is judged as being below the threshold value in Step S21, the feedback control circuit 23 returns to Step S20 and repeats the processing described above. When the detection value is judged as exceeding the predetermined threshold value in Step S21, the flow proceeds to Step S22.

In Step S22, the feedback control circuit 23 first outputs a predetermined gain instruction value to the inversion amplification circuits 13 and 18 and adjusts the gain set to each of these circuits. To control the gains, various control procedures may be employed. For example, the gains of both circuits are increased or decreased or are changed relatively to each other. Various control procedures may be employed in such a fashion as to correspond to the form of practical execution.

When the gain control processing in Step S22 is completed, the feedback control circuit 23 proceeds to the next Step S23, again detects the residual noise component contained in the LPF 21, compares this detection value with the previous detection value and calculates the change rate of the detection value. When the change rate is judged as increasing in the next Step S24, the feedback control circuit 23 returns to Step S22 and repeats the gain control processing. Incidentally, re-control of the gain is of course made in this case in the opposite direction to the direction of previous control from the principle of negative feedback control.

On the other hand, when the change rate of the detection value is judged as decreasing, the feedback control circuit 23 proceeds to Step S25 and judges whether or not the decrement width of the change rate is below a predetermined value stored in advance inside the memory of the feedback control circuit 23. When the decrement width of the change rate does not yet reach the predetermined value, the feedback control circuit 23 returns to Step S22 and repeats the gain control processing. Incidentally, since the change rate of the noise component changes in the decreasing direction, re-control of the gain in this case is made in the direction that further promotes previous control.

When the change rate of the detection value is judged as being below the predetermined value in Step S25, on the other hand, the feedback control circuit 23 proceeds to Step S26, outputs the predetermined delay instruction values to the delay circuits 12 and 17 and controls the delay amounts set to these delay circuits.

To control the delay amounts, various control procedures may be employed. For example, the delay amounts of both circuits are increased or decreased or are changed relatively to each other. Various control procedures may be employed in such a fashion as to correspond to the form of practical execution.

When the control processing of the delay amount in Step S26 is completed, the feedback control circuit 23 proceeds to the next Step S27, again detects the residual noise component contained in the LPF 21, compares this detection value with the previous detection value and calculates the change rate of the detection value. When the change rate is judged as increasing in the next Step S28, the feedback control circuit 23 returns to Step S26 and repeats the delay amount control processing. Incidentally, re-control of the delay amounts is of course made in this case in the opposite direction to the direction of previous control from the principle of negative feedback control.

On the other hand, when the change rate of the detection value is judged as decreasing in Step S28, the feedback control circuit 23 proceeds to Step S29 and judges whether or not the decrement width of the change rate is below a predetermined value stored in advance inside the memory of the feedback control circuit 23. When the decrement width of the change rate is not found as reaching the predetermined value, the gain control circuit 23 returns to Step S26 and repeats the delay amount control processing. Incidentally, since the change rate of the noise component changes in the decreasing direction, re-control of the gain in this case is made in the direction that further promotes previous control.

On the other hand, when the change rate of the detection value is judged as decreasing in Step S29, the feedback control circuit 23 returns to Step S20 as the start of the noise cancellation processing and repeats the processing described above.

Incidentally, the form of the noise cancellation processing in the invention is not limited to the one shown in FIG. 3. For example, the delay amount control processing may be conducted before the gain control processing or may be conducted simultaneously with the gain control processing to detect the residual noise component.

The invention is not limited to the embodiment described above. For example, correction of the sound field may be made, too, by taking the cruising noise inside the cabin into account on the basis of the measurement of the frequency characteristics inside the cabin described already besides the cancellation processing of the cruising noise.

This application is based on Japanese Patent Application No. 2003-168404 which is herein incorporated by reference.

What is claimed is:

1. A noise reduction apparatus in a sound reproduction system for reproducing and outputting sound to a sound field based on electric audio signals, comprising:
first and second microphones arranged in said sound field:
a first signal processing part for executing a delay processing and an inversion amplification processing for a first sound field electric signal acquired through said first microphone on the basis of a first delay instruction value and a first gain instruction value;
a reproduction output part for reproducing and outputting a superimposed signal acquired by superimposing said electric audio signal to an output signal of said first signal processing part as a sound pressure signal;
a second signal processing part for executing a delay processing and an inversion amplification processing for said electric audio signal on the basis of a second delay instruction value and a second gain instruction value;
a signal superimposing part for superimposing a second sound field electric signal acquired through said second microphone with an output signal from said second signal processing part; and
a feedback control part for controlling said first and second delay instruction values and said first and second gain instruction values in accordance with the magnitude of the output signal from said signal superimposing part.

2. A noise reduction apparatus as defined in claim 1, further comprising an initial value setting part for setting an initial value of said first and second delay instruction values and said first and second gain instruction values.

3. A noise reduction apparatus as defined in claim 2, wherein said initial value setting part sets said initial values on the basis of measurement values acquired by measuring an impulse response in said sound field space.

4. A noise reduction apparatus as defined in claim 1, wherein said sound field is inside a cabin of a moving vehicle in which said sound reproduction system is mounted and said second microphone is arranged closer to a driver's seat inside said cabin than said first microphone.
